(19) United States
(12) Patent Application Publication
(10) Pub. No.: US 2012/0283507 A1
(43) Pub. Date: Nov. 8, 2012

(54) Cardiac Assist Devices

(76) Inventor: Theodore J. Lillehei, Minneapolis, MN (US)

(21) Appl. No.: 13/460,837

(22) Filed: Apr. 30, 2012

Related U.S. Application Data
(60) Provisional application No. 61/480,439, filed on Apr. 29, 2011.

(51) Int. Cl. A61M 1/12

(52) U.S. Cl. 600/18

(57) ABSTRACT

One embodiment of the invention provides a cardiac assist device that is in direct blood contact with blood in the heart. It is linked to the heart by surgically coring a hole into the wall tissue of a pumping chamber of the heart, typically the left ventricle. The device includes a contractile element that is linked to the hole in the heart's pumping chamber. The contractile element comprises either an electroactive polymer or (ii) one or more pneumatic or hydraulic bladders. In the contracted state, the contractile element displaces fluid from the pumping chamber of the heart to assist with pumping of the heart and increase the ejection fraction of the pumping chamber.

This application claims priority under 35 U.S.C. 119(e) from U.S. provisional patent application serial no. 61/480,439, filed Apr. 29, 2011.

### BACKGROUND

Congestive heart failure is a debilitating and progressive disease that causes a heart to pump less efficiently over time. Typically, the heart has been weakened by an underlying problem, such as clogged arteries, high blood pressure, a defect in heart muscles or heart valves, or some other medical condition. Many symptoms and conditions associated with heart failure can be treated, but to date in many cases the underlying impairment of the heart cannot.

One characteristic of heart failure is remodeling of the heart—that is, physical change to the size and shape of the heart and thickness of the heart wall. In many cases the wall of the left ventricle thins and stretches in places. The thinned portion of the myocardium is typically functionally impaired and other portions may grow or thicken to compensate.

Typically, the heart enlarges as heart failure progresses, which seems to be the result of the body trying to compensate for weakening heart muscles. The heart can become so enlarged that the heart can no longer provide an adequate supply of blood to the body. As a result, individuals afflicted with congestive heart failure often experience shortness of breath and fatigue even with minimal activity. Also, as the heart enlarges, the heart valves may not adequately close, which further reduces the heart's ability to supply blood to the body.

Drug therapies have been developed to treat individuals afflicted with congestive heart failure. A drug regimen of beta blockers, diuretics, and angiotensin-converting enzyme inhibitors (ACE inhibitors) aims to improve the effectiveness of the heart's contractions and slow CHF progression. Although drug therapy for heart failure can improve the quality of life and also modestly prolong survival, it is well established that many of the currently available approaches do not represent satisfactory long-term treatment options for a large number of patients.

Once the disease progresses to the point that medication is no longer effective, the currently preferred options are a heart transplant or a ventricular assist device (VAD). Approximately 550,000 new cases of CHF are diagnosed in the

United States alone every year. Of these, at least 75,000 individuals are candidates for a heart transplant. But more than 50,000 men and women die every year waiting for a heart transplant because of a lack of donor hearts.

Only a few hundred VADs are implanted in the US each year. VAD use is limited because device implant surgery is highly invasive and complicated. Management of pump volume or pressure is difficult. VAD surgery adds insult to the heart because of the required surgical connections into the ventricle and aorta. But the largest contributor to complications from VAD implantation is the required direct interface of the device with the patient's blood. This can lead to clotting, strokes, and infection.

In addition to drugs, transplants, and VADs, heart failure has been treated with cardiac jackets or restraint devices. These basically consist of flexible material wrapped around the heart. A cardiac jacket is fitted around an enlarged heart to physically limit expansion of the heart during diastole. This may prevent further enlargement of the heart.

Improved methods and devices for treating heart failure and other cardiac diseases are needed.

### SUMMARY

One embodiment of the invention provides a cardiac assist device that is in direct blood contact with blood in the heart. It is linked to the heart by surgically coring a hole into the wall tissue of a pumping chamber of the heart, typically the left ventricle.

The device includes a contractile element that is linked to the hole in the heart's pumping chamber. The contractile element comprises either an electroactive polymer or (ii) one or more pneumatic or hydraulic bladders.

Thus, one embodiment of the invention provides a cardiac assist device comprising: (1) a contractile element selected from the group consisting of (a) a fabric patch comprising or linked to an electroactive polymer, and (b) one or more pneumatic or hydraulic bladders. The contractile element is adapted to be attached directly or indirectly to cardiac wall tissue surrounding a hole in the cardiac wall by surgically penetrating the wall of a pumping chamber of the heart to form a hole in the wall of the pumping chamber and attaching the contractile element directly or indirectly to cardiac tissue of the wall surrounding the hole. The cardiac assist device also comprises, linked to the contractile element, (2) a means for contracting the contractile element. The contractile element in its contracted state reduces the volume of the pumping chamber and the device is adapted to contract the contractile element in a propulsate manner.

### BRIEF DESCRIPTION OF THE DRAWINGS

FIG. 1 is a schematic drawing showing a heart and a hole cored into the left ventricle (LV) near the apex of the heart to attach a device of the invention to the hole 1. In the figures, LA and RA refer to the left and right atrium, respectively, and LV and RV refer to the left and right ventricle respectively.

FIG. 2 shows a contractile element 2 of a device of the invention in relaxed state position 2a (solid lines), generally coplanar with the wall of the left ventricle, and in position 2b (dotted lines), the contracted state, where the contractile element 2 forms a bubble extending inward from the wall of the pumping chamber (left ventricle) to displace a portion of the fluid volume of the left ventricle.

FIG. 3 shows an alternative embodiment of a device of the invention where the contractile element 2 forms a pouch extending outward from the wall of the pumping chamber in relaxed state 2a, and in the contracted state 2b the pouch decreases in volume, as shown in position 2b (dotted line).

In FIG. 4 shows another embodiment where the contractile element 2 pulls the wall of the left ventricle tighter (to the dotted line position) to displace fluid from the left ventricle and assist contraction of the left ventricle.

FIG. 5 shows another embodiment of a device of the invention comprising a generator electrically linked to the contractile element 2.

FIG. 6 shows another embodiment of the invention where the contractile element 2 comprises a pneumatic bladder 22.

FIG. 7 shows an embodiment of the invention involving an apex-to-descending-aorta conduit.

FIG. 8 shows an embodiment of the invention involving a cardiac jacket comprising pneumatic or hydraulic chambers as a contractile element, where the jacket is adapted to raise and deform the apex of the heart in the jackets contracted state, and thereby reduce the volume of the left ventricle to assist pumping of the heart.

FIG. 9 shows another embodiment of a device of the invention with a contractile element 2 comprising a pneumatic bladder 22 attached to a hole cored in a pumping chamber near the apex of the heart to assist pumping of the heart.

### DETAILED DESCRIPTION

The term “propulsate manner” is used herein to mean that the contraction of the contractile element occurs during systole, i.e., moves blood in synchrony with contraction of the heart moving blood.

The term “contracted state” is used herein to mean the position of the contractile element that displaces blood from the heart, where the contractile element is linked to the heart, or from a conduit where the contractile element is linked to a conduit. Where the contractile element includes a pneumatic or hydraulic bladder, the contracted state is the state where the bladder is relatively filled with fluid, and the relaxed state is the state where the bladder is relatively emptied of fluid. Where the contractile element comprises an electroactive polymer (EAP), the EAP changes shape in response to application of an electric field, and this moves the contractile element. The contracted state with an EAP may be either the state where the EAP is exposed to a stronger electric field or that where it is exposed to a weaker or no electric field.

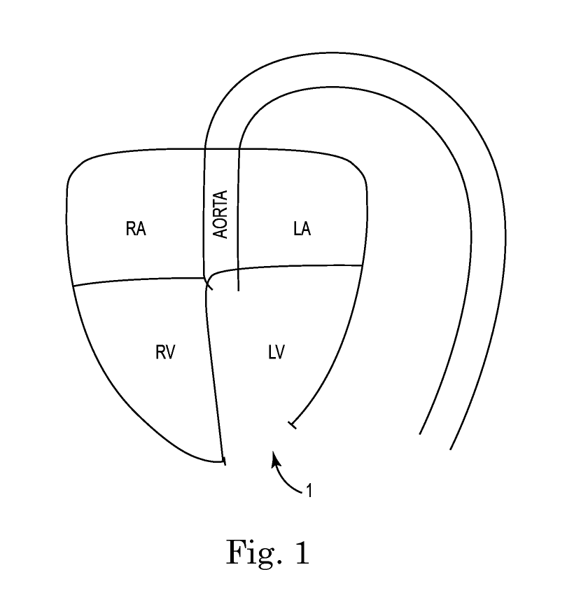

FIG. 1 is a schematic of a heart, showing a surgically created hole 1 in the wall of the left ventricle, the hole 1 being located at the apex of the heart. In the figures, LA and RA refer to the left and right atrium, respectively, and LV and RV refer to the left and right ventricle respectively.

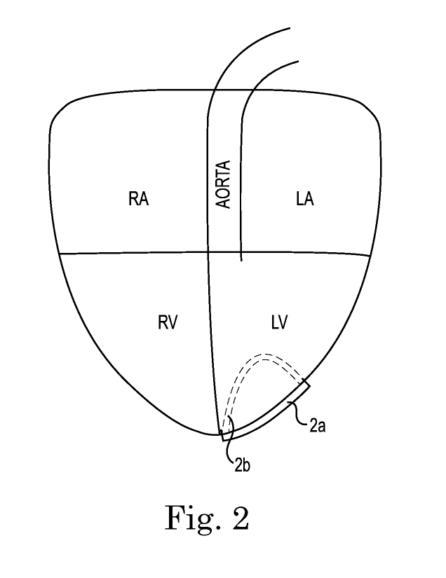

FIG. 2 shows a contractile element 2 in relaxed state position 2a (solid lines), generally coplanar with the wall of the left ventricle, and in position 2b (dotted lines), the contracted state, where the contractile element 2 forms a bubble extending inward from the wall of the pumping chamber (left ventricle) to displace a portion of the fluid volume of the left ventricle.

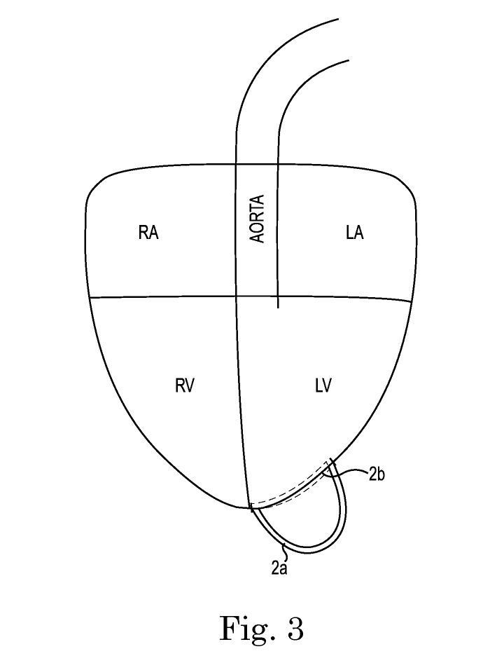

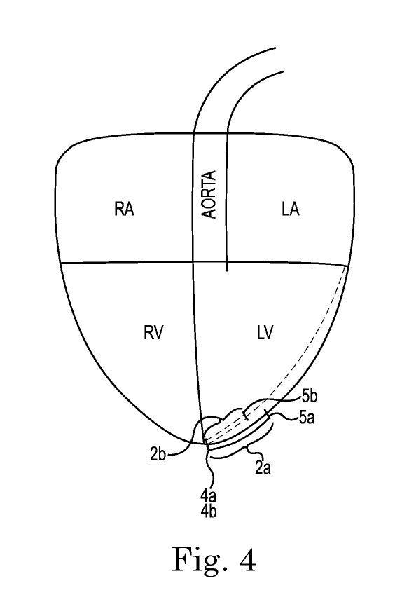

FIG. 3 shows an alternative embodiment where the contractile element 2 forms a pouch extending outward from the wall of the pumping chamber in relaxed state 2a, and in the contracted state 2b the pouch decreases in volume, in some cases disappearing as the contractile element inner surface is generally coplanar with the wall of the left ventricle, as shown in FIG. 3 by element 2b (dotted line).

In FIG. 4 the contractile element forms a disc attached about the periphery of the disc directly or indirectly to cardiac wall tissue surrounding the hole of the cardiac wall, wherein in the contracted state the contractile device pulls closer together the cardiac tissue surrounding the hole to decrease cross-sectional area of the hole and thereby decrease the volume of the pumping chamber. In contracted position 2b (dotted lines), the diameter of the contractile element 2b is smaller than in relaxed position 2a (solid line). This results in pulling together the cardiac tissue 4 and 5 at opposite ends of the hole. In the contracted position the distance between points 4b and 5b is closer than the distance between points 4a and 5a in the relaxed position. And this pulls the wall of the left ventricle inward in the contracted position to reduce the volume of the left ventricle.

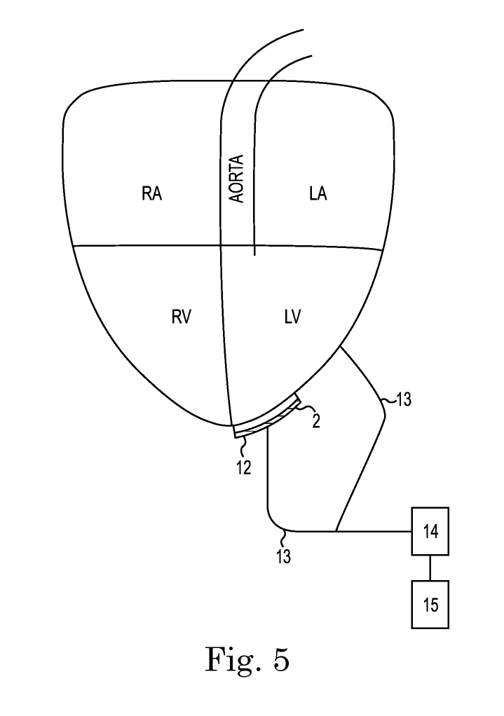

The device may further comprise a computerized generator 14 linked by one or more electrical leads 13 to the electroactive polymer of the contractile element, as shown in FIG. 5.

The generator 14 may be linked to any suitable power source. But preferably the device includes a battery 15 electrically coupled to the generator. With a battery, the device can be entirely implanted, and the patient is not tethered to any external components and can move on his own. Having all components of the device internal to the patient also reduces the risk of infection.

The computerized generator, battery, and electrical leads essentially are a pacemaker. The pacemaker stimulates the electroactive polymers to stimulate contraction of the polymers, which assists contraction of the heart. The pacemaker may also stimulate the heart in coordination with stimulating the EAP, in which case the pacemaker includes one or more electrical leads linking the generator to one or more electrodes contacting cardiac muscle of one or more pumping chambers of the heart to pace pumping of the heart muscle. The pacemaker is preferably rate responsive. That is, the rate of pacing is responsive to physiological signals, including the patient's natural heart rate or breathing rate, or responsive to movement of the patient. In FIG. 5 the electrical lead 13 is shown linked to both EAP 12 of the contractile element 2 and to heart muscle.

Where the contractile element of the device comprises an electroactive polymer (EAP) (element 12 in FIG. 5), the EAP changes shape in response to an electric current or potential, as described in U.S. patent application Ser. No. 12/590,378 and references cited therein. The contractile element may comprise a fabric sheet that covers the hole in the cardiac wall. The fabric sheet may be composed of or comprise EAP or may be linked at certain points to EAP

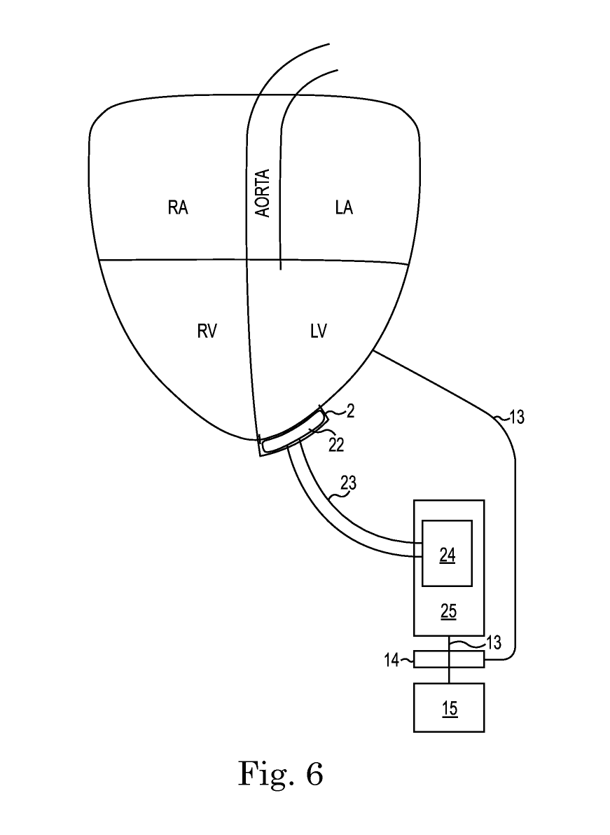

In another embodiment shown in FIG. 6 the contractile element comprises one or more pneumatic or hydraulic bladders. The one or pneumatic or hydraulic bladders 22 are functionally linked by a tube 23 to a reservoir 24, which in turn is linked to a pump 25 for pumping fluid from the reservoir 24 to the hydraulic or pneumatic bladder 22. A power pack 15 powers the pump 25. A generator 14 may also be linked to the pump 25 by electrodes 13 to pace the pump or valves of the pump. The generator 14 may also be linked by electrodes 13 to the heart muscle to pace the heart to pump synchronously with the pump 25. Again, the power pack 15, computerized generator 14, and electrodes 13 may be considered a pacemaker.

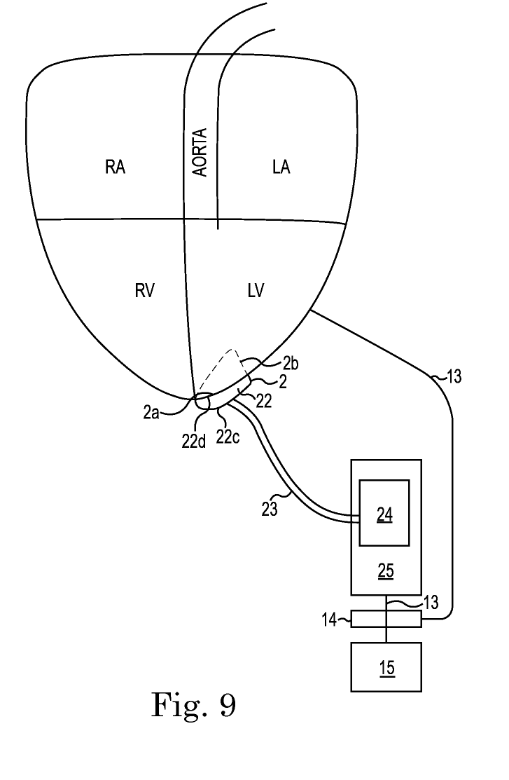

In a specific embodiment shown in FIG. 9, the bladder 22 includes an outer wall 22c and an inner wall 22d, and the outer wall 22c is less expandible than the inner wall 22d, i.e., the outer wall 22c is made of a more rigid or stronger material than the inner wall 22d. When the bladder 22 expands from the relaxed state to the contracted state, the inner wall 22d expands from position 2a to position 2b to displace blood, while the outer wall 22c does not move as much.

In other embodiments, the contractile element 22 includes a rigid or semirigid casing about the outside of the bladder to prevent its expansion outward from the hole 1 and force any expansion of the bladder or bladders to occur toward the interior of the ventricle to displace blood from the ventricle.

The power pack 15 and pump 25 may be internal or external to the body. If external, they may be in a wearable vest, and the tube 23 passes through the body wall, preferably in the abdomen. A percutaneous portal such as is described in U.S. patent publication No. 20080281147 may be used.

Where the contractile element of the present devices comprises an EAP, the cardiac assist device is preferably entirely internal to the body. However, in some embodiments the battery or power pack may be external.

In one embodiment, the surgically created hole in the heart is located at the apex of the heart.

In an alternative embodiment, the contractile element is adapted to be attached directly or indirectly to cardiac wall tissue surrounding a hole of the cardiac wall by surgically penetrating the wall of a pumping chamber of the heart by removing necrotic or damaged tissue in the wall of a pumping chamber at the site of a myocardial infarction to form a hole in the wall of the pumping chamber and attaching the contractile element directly or indirectly to cardiac tissue of the wall surrounding the hole. Since the tissue at the site of a myocardial infarction is damaged, this may be the best tissue to remove and replace with the contractile element.

In another embodiment, the cardiac device comprises an inner surface covering the hole in the cardiac wall and in contact with blood in the heart pumping chamber, wherein the inner surface comprises a material that promotes tissue growth. Tissue growth will make the surface a natural surface to the body, and thus less prone to promote clot formation or adherence. Materials that promote tissue growth are known in the art, and include, e.g., polyurethane. Growth factors and other growth-promoting substances may also be impregrated in the surface and may be the materials that promote tissue growth.

Another embodiment of the invention provides a cardiac assist device comprising: (a) a heart-apex-to-descending-aorta conduit adapted to link the left ventricle of a heart to the descending aorta and adapted to be connected by (i) attaching a proximal end of the conduit directly or indirectly to cardiac wall tissue surrounding a hole in the cardiac wall by surgically penetrating the wall of the left ventricle at the apex of the heart to form a hole in the wall of the left ventricle at the apex and attaching the proximal end of the conduit directly or indirectly to cardiac tissue of the wall surrounding the hole in the left ventricle; and (ii) attaching a distal end of the conduit directly or indirectly to aortic wall tissue surrounding a hole in the wall of the descending aorta by surgically penetrating the wall of the descending aorta to form a hole in the wall of the descending aorta and attaching the distal end of the conduit directly or indirectly to tissue of the wall surrounding the hole in the descending aorta; and (b) a contractile element selected from the group consisting of (a) a contractile structure comprising an electroactive polymer and (b) one or more pneumatic or hydraulic bladders; the contractile element functionally linked to the conduit to assist pumping blood through the conduit from the left ventricle to the descending aorta in a propulsatile manner; linked to (c) a means for contracting the contractile element.

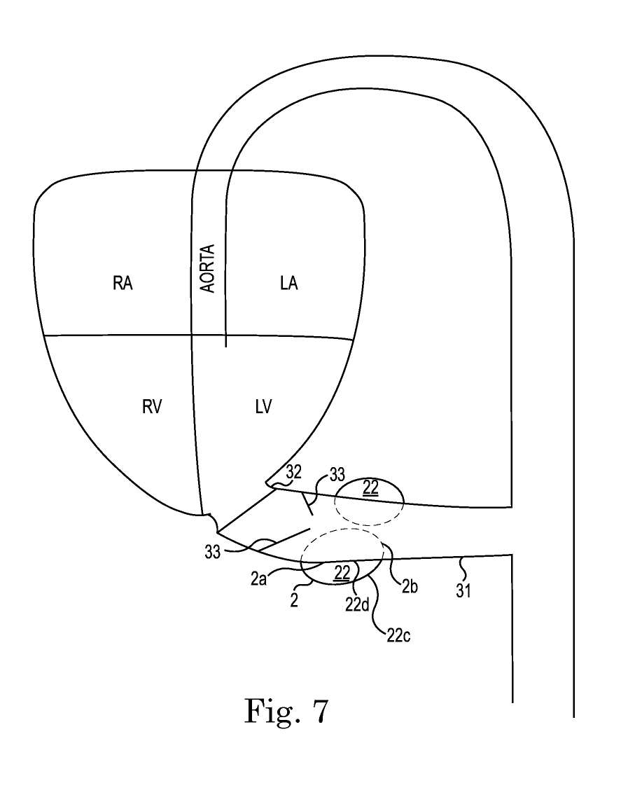

In FIG. 7, the heart-apex-to-descending-aorta conduit 31 is linked to the apex of the heart indirectly through sewing ring 32. Sewing ring 32 is sutured directly to the cardiac wall tissue surrounding the hole at the apex of the heart. The other end of the conduit 31 is sutured to the wall tissue of the descending aorta surrounding a hole surgically created in the descending aorta. Contractile element 2 is two pneumatic or hydraulic bladders 22 positioned on opposite sides of the conduit 31. (The two bladders 22 could also be a single bladder forming a ring around the circumference of the conduit.) In relaxed state 2a, the bladders 22 are relatively empty and allow the conduit to expand. In contracted state 2b (shown by dotted lines), the bladders expand and thereby displace blood volume in the conduit. If the conduit does not contain a valve, the blood is forced both downstream into the descending aorta and upward into the left ventricle. In other embodiments, the conduit contains a valve 33. The valve 33 may be to the heart side of the contractile element 2. When the contractile element 2 contracts to push blood (expanded bladder position 2a) the contractile element pushes blood in the conduit toward the descending aorta, and the valve prevents blood flow in the opposite direction toward the heart.

In specific embodiments, the outer wall 22c of the bladder 22 is made of a stronger or more rigid material than the inner wall 22d. When the bladder expands, the inner wall 22d expands to position 2b, while the outer wall 22c maintains more constant dimensions and does not move as much. In other embodiments, the contractile element 2 includes a rigid or semirigid casing about the outside of the bladder to prevent its expansion outward from the conduit and force any expansion of the bladders to occur toward the interior of the conduit and thereby decrease the conduit diameter.

Where the contractile element is or includes one or more pneumatic or hydraulic bladders, in one embodiment, the bladders are placed inside of the conduit, in a similar manner to an intraaortic balloon pump. The bladders may be threaded into the conduit in this case through the femoral artery. In other embodiments, the bladders are placed into the conduit through the wall of the conduit.

In other embodiments, the bladders surround the wall of the conduit and in the contracted state expand to push against the walls of the conduit and narrow the conduit, forcing blood out of the conduit. In other embodiments, the inner wall of the bladder may form an inner wall of the conduit, as is shown in FIG. 7.

The conduit may be composed of any biocompatible material, such as GORE-TEX, TEFLON, nylon, polyurethane, or other suitable materials.

Another embodiment provides a cardiac assist device comprising a cardiac jacket adapted to fit generally around at least a portion of the heart. The jacket comprises an apical contractile element selected from the group consisting of (a) a contractile structure comprising an electroactive polymer, and (b) one or more pneumatic or hydraulic bladders. The apical contractile element is adapted to be held in the jacket over the apex of the heart, and is linked to a means for contracting the apical contractile element. The apical contractile element has a contracted and a relaxed state and in its contracted state deforms and raises the apex of the heart and reduces the volume of the left ventricle, and the device is adapted to contract the apical contractile element in a propulsate manner.

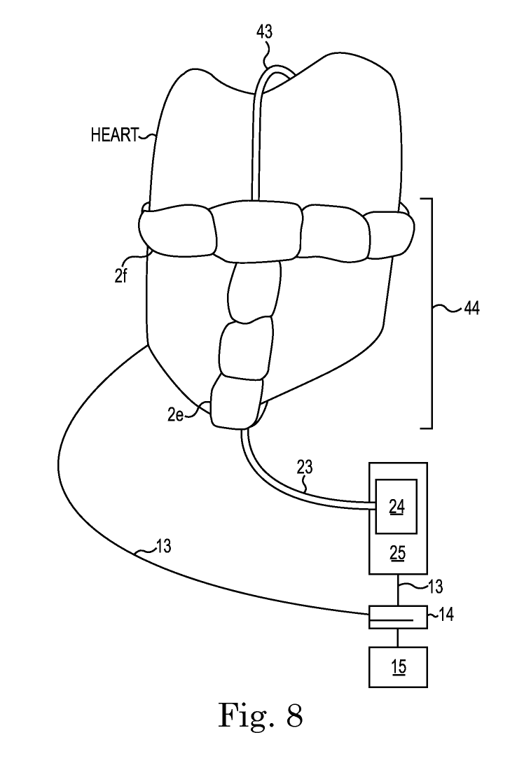

An example of this embodiment is shown in FIG. 8. Pneumatic or hydraulic bladders 2F around the equator and 2E at the apex together form a cardiac jacket 44. The bladders are linked by a tube 23 to a pump 25, which is controlled by computerized generator 14 and electrodes 13 and power pack 15 to pace the contractile elements 2E and 2F in a propulsate manner. To maintain the jacket in position, the jacket may also include a strap 43 over the base of the heart.

In FIG. 8, the bladders form the jacket. In other embodiments, the jacket may include an outer shell that provides a restraint to heart expansion and holds the contractile elements or may be considered to form a portion of the contractile elements. The shell holding the contractile element could be a fabric jacket or a metal or plastic mesh, for instance. Similar jackets have been manufactured by Paracor Medical Inc. and Acorn Cardiovascular. The shell may be composed of any biocompatible material, including polymers, stainless steel, or nitinol. It may be nonexpandible or may provide some elasticity.

In some embodiments, the contractile element is a contractile structure comprising an EAP. Cardiac jackets with EAPs are disclosed in U.S. patent application Ser. No. 12/590,378.

In some embodiments, the cardiac jacket further comprises in addition to the apical contractile element an equatorial contractile element selected from the group consisting of (a) a contractile structure comprising an electroactive polymer, and (b) one or more pneumatic or hydraulic bladders. The equatorial contractile element is adapted to be held in the jacket generally equatorially against or around the heart and is linked to a means for contracting the equatorial contractile element. The equatorial contractile element in its contracted state reduces the equatorial diameter of the left ventricle and reduces the volume of the left ventricle, and the device is adapted to contract the equatorial contractile element in a propulsate manner.

Both the apical and equatorial contractile elements can comprise independently an EAP or pneumatic or hydraulic bladders.

In the devices described herein comprising pneumatic or hydraulic bladders, the pneumatic gas can be air, nitrogen, argon, or helium, or other suitable gas. The hydraulic fluid is preferably saline, so that a leak would have minimal impact on the patient.

Additional information concerning the design of cardiac assist devices that is applicable also to the devices described herein can be found in U.S. patent application Ser. No. 13/304,277, filed Nov. 23, 2011.

All patents, patent documents, and other references cited are incorporated by reference.

What is claimed is:

1. A cardiac assist device comprising:
a contractile element comprising (a) a fabric patch comprising or linked to an electroactive polymer, or (b) one or more pneumatic or hydraulic bladders; the contractile element adapted to be attached directly or indirectly to cardiac wall tissue surrounding a hole in the cardiac wall by surgically penetrating the wall of a pumping chamber of the heart to form a hole in the wall of the pumping chamber and attaching the contractile element directly or indirectly to cardiac tissue of the wall surrounding the hole; linked to
means for contracting the contractile element;
wherein the contractile element has a contracted and a relaxed state and in its contracted state reduces the volume of the pumping chamber and the device is adapted to contract the contractile element in a propulsate manner.

2. The cardiac assist device of claim 1 wherein the contractile element comprises one or more hydraulically or pneumatically operated bladders.

3. The cardiac assist device of claim 1 wherein the contractile element comprises a fabric patch comprising or linked to an electroactive polymer.

4. The cardiac assist device of claim 1 wherein the contractile element forms a pouch extending outward from the wall of the pumping chamber, and in the contracted state the pouch decreases in volume.

5. The cardiac assist device of claim 1 wherein the contractile element forms a surface that in the contracted state forms a bubble expanding inward from the wall of the pumping chamber to displace a portion of inner fluid volume of the pumping chamber.

6. The cardiac device of claim 1 wherein the contractile device forms a disc attached about the periphery of the disc directly or indirectly to cardiac wall tissue surrounding the hole of the cardiac wall, wherein in the contracted state the contractile device pulls closer together the cardiac tissue surrounding the hole to decrease cross-sectional area of the hole and thereby decrease the volume of the pumping chamber.

7. The cardiac device of claim 1 wherein the hole is located at the apex of the heart.

8. The cardiac device of claim 1 wherein the contractile element is adapted to be attached directly or indirectly to cardiac wall tissue surrounding a hole of the cardiac wall by surgically penetrating the wall of a pumping chamber of the heart by removing necrotic or damaged tissue in the wall of a pumping chamber at the site of a myocardial infarction to form a hole in the wall of the pumping chamber and attaching the contractile element directly or indirectly to cardiac tissue of the wall surrounding the hole.

9. The cardiac device of claim 1 wherein the cardiac device comprises an inner surface covering the hole in the cardiac wall and in contact with blood in the heart pumping chamber, wherein the inner surface comprises a material that promotes tissue growth.
